(19) United States
(12) Patent Application Publication
(10) Pub. No.: US 2024/0018231 A1
(43) Pub. Date: Jan. 18, 2024

(54) INTRANASAL IMMUNOTHERAPT FOR THE TREATMENT OF ALZHEIMER'S DISEASE

(71) Applicant: Rush University Medical Center, Chicago, IL (US)

(72) Inventor: Kalipada Pahan, Skokie, IL (US)

(21) Appl. No.: 18/250,890

(22) PCT Filed: Oct. 27, 2021
(86) PCT No.: PCT/US2021/056801

§ 371 Date: Apr. 27, 2023

Related U.S. Application Data
(60) Provisional application No. 63/107,496, filed on Oct. 30, 2020.

(51) Int. Cl. C07K 16/24; A61K 38/20; A61P 25/28

(52) U.S. Cl. CPC C07K 16/244; A61K 38/208; A61P 25/28; A61K 2039/505

(57) ABSTRACT

The present disclosure generally relates to pharmaceutical compositions useful for the treatment of diseases and disorders. More particularly, the disclosure relates to immunotherapy comprising intranasal immunotherapy with neutralizing monoclonal antibodies (mAbs) against IL-12 p40 homodimer for the treatment of an Alzheimer's disease.

### CROSS-REFERENCE TO RELATED APPLICATIONS

The present application claims the benefit of U.S. Provisional Patent Application No. 63/107,496, filed Oct. 30, 2020, the contents of which are incorporated into the present application in their entirety.

### FIELD OF THE INVENTION

The present disclosure generally relates to pharmaceutical compositions useful for the treatment of diseases and disorders. More particularly, the disclosure relates to immunotherapy comprising intranasal immunotherapy with neutralizing monoclonal antibodies (mAbs) against IL-12 p40 homodimer for the treatment of an Alzheimer's disease.

### BACKGROUND

Alzheimer's disease is progressive neurodegenerative disease with classic memory impairment and cognitive disorder. The pathological hallmarks of Alzheimer's disease are presence of senile plaques (SPs), composed of oligomeric amyloid beta (Aβ40/42) and formation neurofibrillary tangles (NFTs), originating from Tau hyper-phosphorylation, in the cortex and hippocampus of brain. See Takahashi et al., “Co-occurrence of Alzheimer's disease β-amyloid and tau pathologies at synapses,” (2010) Neurobiol Aging 31, 1145-1152; Li et al., “The role of intracellular amyloid beta in Alzheimer's disease,” (2007) Prog Neurobiol 83, 131-139.

It is widely believed that AD is a multifactorial disorder affected by a mix of genetic, environmental, and lifestyle factors. However, the abnormal accumulation of Aβ and formation NFTs induces neuro-inflammation and subsequent neuronal loss, which is the primary cause of Alzheimer's disease. See Citron, M., Teplow, D. B., and Selkoe, D. J. (1995) Generation of amyloid beta protein from its precursor is sequence specific. Neuron 14, 661-670

Many types of treatments have been tried to minimize or reverse the impairments of this disease. Most of the established treatments that are used today try to counterbalance the neurotransmitter imbalance of the disease. The acetylocholinesterase inhibitors (AChEIs) which are approved for the treatment of AD are donepezil, galantamine, and rivastigmine. Their development was based in the original cholinergic hypothesis which suggests that the progressive loss of limbic and neocortical cholinergic innervation in AD is critically important for memory, learning, attention, and other higher brain functions decline. Furthermore, neurofibrillary degeneration in the basal forebrain is probably the primary cause for the dysfunction and death of cholinergic neurons in this region, giving rise to a widespread presynaptic cholinergic denervation. The AChEIs increase the availability of acetylcholine at synapses and have been proven clinically useful in delaying the cognitive decline in AD. See for example Yiannopoulou et al., “Current and Future Treatments in Alzheimer Disease: An Update,” (2020) J Cent Nery Syst Dis. 12: 1179573520907397.

Other approaches besides AChEIs have also been used. For example, the low-to-moderate affinity noncompetitive N-methyl-d-aspartate (NMDA) receptor antagonist, memantine, is approved for moderate to severe AD. Id. Memantine binds preferentially to open NMDA receptor—operated calcium channels blocking NMDA-mediated ion flux and ameliorating the dangerous effects of pathologically elevated glutamate levels that lead to neuronal dysfunction. Id.

In clinical trials, both Aβ and tau are prime targets for disease-modifying treatments (DMTs) in AD. From this point of view, AD could be prevented or effectively treated by decreasing the production of Aβ and tau; preventing aggregation or misfolding of these proteins; neutralizing or removing the toxic aggregate or misfolded forms of these proteins; or a combination of these modalities. Id.

There also has been extensive research in the exploration of the inflammatory mechanisms in AD disease. Studies have shown that activation of glial cells, microglia, and astrocytes induces the production of inflammatory cytokines, mainly interleukin 1β (IL-1β) and tumor necrosis factor α (TNF-α). TNF-α signaling has been proved to exacerbate both Aβ aggregation and tau phosphorylation in vivo, whereas its levels have been found elevated in brain and plasma of patients with AD. See Alam et al., “Inflammatory process in Alzheimer's and Parkinson's diseases: central role of cytokines,” (2016) Curr Pharm Des. 22:541-548; Chang et al., “Tumor necrosis factor α inhibition for Alzheimer's disease,” (2017) J Cent Nery Syst Dis. 9:1179573517709278.

However, to date, despite intense investigation, no effective therapy is available against AD. Therefore, developing an effective neuroprotective therapeutic approach to slow down or halt the disease progression are of paramount importance. The present disclosure addresses this need.

### SUMMARY OF THE DISCLOSURE

The p40 family of cytokines has four members which include interleukin-12 (IL-12), the p40 monomer (p40), the p40 homodimer (p40₂), and IL-23. Previously, with the knowledge that the heterodimers rule and the homodimers remain as mere spectators, those skilled in the art thought that only IL-23 and IL-12 were endowed with biological functions. Both p40₂ and p40 were considered as inactive molecules with unknown functions. In WO2012159100, the current inventor demonstrated that neutralizing monoclonal antibodies (mAb) against mouse p40₂ and p40, including recombinant p40 and/or monoclonal antibody against p40₂ (mAb-p40₂ a3-1d), are useful in the treatment of multiple sclerosis and rheumatoid arthritis.

Intranasal drug administration has been shown to offer many advantages over standard systemic delivery systems, such as its non-invasive character, a fast onset of action and in many cases reduced side effects due to a more targeted delivery. Intranasal drug delivery has been suggested to be a particularly interesting delivery route for the treatment of neurological or neurodegenerative disorders. Systemic approaches often fail to efficiently supply the CNS with drugs. See for example Keller et al., “Intranasal drug delivery: opportunities and toxicologic challenges during drug development,” (2021) Drug Delivery and Translational Research https://doi.org/10.1007/s13346-020-00891-5.

The inventors have discovered that utilizing a mouse model for AD in 5XFAD mice, treatment with p40₂ (mAb-p40₂ a3-1d), at a very low dose via intranasal route is capable of reducing the load of amyloid plaques from the hippocampus. Thus, the present disclosure relates to methods and pharmaceutical compositions and/or formulations useful for the treatment of Alzheimer's disease and related disorders in which the amyloid deposition is present in the brain. More particularly, the present disclosure relates to methods and pharmaceutical compositions and/or formulations comprising intranasally administering to a patient in need thereof an effective amount of a pharmaceutical composition comprising p40₂ (mAb-p40₂ a3-1d) antibodies.

In some embodiments, a treatment for AD is disclosed which includes administration to a patient of at least one monoclonal antibody against p40₂ (mAb-p40₂ a3-1d).

In another embodiment, a method for reducing the load of amyloid plaques in the brain is disclosed which includes administration to a patient of at least one monoclonal antibody against p40₂ (mAb-p40₂ a3-1d).

In yet another embodiment, a treatment for Alzheimer's disease is disclosed which includes administration to a patient of a combination of recombinant p40 and at least one monoclonal antibody against p40₂ (mAb-p40₂ a3-1d).

In still another embodiment, a method for reducing the load of amyloid plaques in the brain is disclosed which includes administration to a patient of a combination of recombinant p40 and at least one monoclonal antibody against p40₂ (mAb-p40₂ a3-1d).

In any of the embodiments, the pharmaceutical composition is administered to the patient by injection, orally, with a transdermal patch, and/or intranasally. In some embodiments, the pharmaceutical composition is preferably inhaled by the patient.

In other embodiments, the pharmaceutical composition is formulated together with a pharmaceutically acceptable carrier or excipient. For example, in some embodiments, the pharmaceutical composition is formulated together with saline.

In any embodiment, the desired treatment can be administered as a single dose. In other embodiments, the desired treatment is administered in a plurality of doses.

These and other embodiments and features of the disclosure will become more apparent through reference to the following description, the accompanying figures, and the claims. Furthermore, it is to be understood that the features of the various embodiments described herein are not mutually exclusive and can exist in various combinations and permutations.

### BRIEF DESCRIPTION OF THE DRAWINGS

FIG. 1A-D shows the results of weekly intranasal treatment with p40₂mAba3-1d reduces the levels of Aβ1-40 and Aβ1-42 in serum and hippocampus of 5XFAD mice. Six-month old 5XFAD Tg mice (n=6 per group) were treated with p40₂ mAba3-1d (25 ng/mouse/week) intranasally once a week. Briefly, 25 ng p40₂ mAb was dissolved in 2 μl normal saline, mice were hold in supine position and 1 μl volume was delivered into each nostril using a pipette. Control 5XFAD mice received only 2 μl saline as vehicle. After 30 d of treatment (five weekly doses), ELISA quantification of Aβ1-40 (FIG. 1A and FIG. 1C) and Aβ1-42 (FIG. 1B and FIG. 1D) was performed in serum (FIG. 1A and FIG. 1B) and (TBS+Triton X-100) (FIG. 1C and FIG. 1D) extracted hippocampal tissues. Results are mean+SEM of six mice per group. ***p<0.001; **p<0.01; *p<0.05.

FIG. 2A-E shows the results of weekly intranasal treatment with p40₂ mAba3-1d improves spatial learning and memory of 5XFAD mice. Six-month-old 5XFAD Tg mice (n=6 per group) were treated with p40₂ mAb a3-1d (25 ng/mouse/week) intranasally once a week. Briefly, ng p40₂ mAb was dissolved in 2 μl normal saline, mice were hold in supine position and 1 μl volume was delivered into each nostril using a pipette. Control 5XFAD mice received only 2 μl saline as vehicle. After 30 d of treatment (five weekly doses), mice were tested for Barnes maze (FIG. 2A, Heat-map; FIG. 2B, Latency; FIG. 2C, Error) and T maze (FIG. 2D, Positive turn; FIG. 2E, Negative turn). Results are mean+SEM of six mice per group. ***p<0.001; **p<0.01; *p<0.05.

FIG. 3A-E depicts that weekly intranasal treatment with p40₂ mAb a3-1d does not modulate locomotor activities in 5XFAD mice. Six-month old 5XFAD Tg mice (n=6 per group) were treated with p40₂ mAb a3-1d (25 ng/mouse/week) intranasally once a week. Briefly, 25 ng p40₂ mAb was dissolved in 2 μl normal saline, mice were hold in supine position and 1 μl volume was delivered into each nostril using a pipette. Control 5XFAD mice received only 2 μl saline as vehicle. After 30 d of treatment (five weekly doses), mice were tested for locomotor activities (FIG. 3A, open field heat-map; FIG. 3B, total distance; FIG. 3C, moving time; FIG. 3D, velocity; FIG. 3E, rest time). Results are mean+SEM of six mice per group. NS, not significant.

### DETAILED DESCRIPTION

Throughout this disclosure, various quantities, such as amounts, sizes, dimensions, proportions, and the like, are presented in a range format. It should be understood that the description of a quantity in range format is merely for convenience and brevity and should not be construed as an inflexible limitation on the scope of any embodiment. Accordingly, the description of a range should be considered to have specifically disclosed all the possible subranges as well as all individual numerical values within that range unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 4.62, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “includes”, “comprises”, “including” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C).

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

The present disclosure is based on the discovery that the inventors have utilized a mouse model for AD in 5XFAD mice, treatment with p40₂ (mAb-p40₂ a3-1d), at a very low dose via intranasal route is capable of reducing the load of amyloid plaques from the hippocampus. Thus, the present disclosure relates to methods and pharmaceutical compositions and/or formulations useful for the treatment of Alzheimer's disease and related disorders in which the amyloid deposition is present in the brain. More particularly, the present disclosure relates to methods and pharmaceutical compositions and/or formulations comprising intranasally administering to a patient in need thereof an effective amount of a pharmaceutical composition comprising p40₂ (mAb-p40₂ a3-1d) antibodies.

The p40 family of cytokines has four members which include interleukin-12 (IL-12), the p40 monomer (p40), the p40 homodimer (p40₂), and IL-23. Previously, with the knowledge that the heterodimers rule and the homodimers remain as mere spectators, those skilled in the art thought that only IL-23 and IL-12 were endowed with biological functions. Both p40₂ and p40 were considered as inactive molecules with unknown functions. In WO2012159100, the current inventor demonstrated that neutralizing monoclonal antibodies (mAb) against mouse p40₂ and p40, including recombinant p40 and/or monoclonal antibody against p40₂ (mAb-p40₂ a3-1d), are useful in the treatment of multiple sclerosis and rheumatoid arthritis.

Intranasal Compositions

Intranasal drug administration has been shown to offer many advantages over standard systemic delivery systems, such as its non-invasive character, a fast onset of action and in many cases reduced side effects due to a more targeted delivery. Intranasal drug delivery has been suggested to be a particularly interesting delivery route for the treatment of neurological or neurodegenerative disorders. Systemic approaches often fail to efficiently supply the CNS with drugs. See for example Keller et al., “Intranasal drug delivery: opportunities and toxicologic challenges during drug development,” (2021) Drug Delivery and Translational Research https://doi.org/10.1007/s13346-020-00891-5.

In some embodiments, the pharmaceutical composition may be administered to a patient as nasal drop (intranasally) or using a nebulization technique. A nebulizer may be used to change a liquid solution of a pharmaceutical composition into a fine mist that may be inhaled by a patient. The inventor determined numerous benefits of these techniques.

For example, the dosage of the pharmaceutical composition can be significantly decreased when either nasal drop or nebulization is used as the delivery method. In some instances, the dosage may be reduced by about one tenth or one twentieth as compared to, for example, injections, oral administration/ingestion of a liquid solution or oral administration/ingestion of a pill. Moreover, using a nebulization technique or nasal drop bypasses the digestive system whereas ingesting a pill or liquid solution of a pharmaceutical composition sends the composition to the digestive system. For example, diarrhea is a common side effect in some urea cycle disorder patients taking glycerol phenylbutyrate orally. Such side effect will be avoided by intranasal administration of glycerol phenylbutyrate and glycerol tribenzoate. Finally, using either a nasal drop or nebulization technique allows the pharmaceutical composition to travel from the olfactory bulb directly to the brain.

In some embodiments, the nebulized pharmaceutical composition may be inhaled through one or both of the mouth or the nasal passage. Without being bound to any theory, it is believed that nasal administration of the composition can take advantage of “nose-to-brain” (N2B) transport systems in which several possibilities exist for bypassing the blood-brain-barrier for direct delivery to the brain. These include the draining of drugs absorbed in the nasal mucosa into the sinus and eventually to the carotid artery, where a “counter-current transfer” from venous blood to the brain may occur. Lymphatic drainage into the perivascular space from the olfactory trigeminal nerves between the central nervous system (CNS) have also been postulated as the mechanism of N2B transport.

Nebulizers are known in the art and the invention of the present disclosure can be used in connection with any nebulizer. For example, the pharmaceutical composition disclosed herein may be nebulized with an inhaler or a Buxco® Inhalation Tower All-In-One Controller.

“Effective or Therapeutic Amount”

Effective or therapeutic amounts of the compositions of this disclosure include any amount sufficient to treat or inhibit the progression of AD. In other embodiments, the effective amounts of the compositions include any amount sufficient to diminish or clear a particular brain region of amyloid plaques. In still other embodiments, effective amounts of the compositions include any amount sufficient to improve learning and spatial memory.

The amount of active ingredient that may be combined with the optional carrier materials to produce a single dosage form may vary depending upon the host treated and the particular mode of administration. The specific dose level for any particular patient may depend upon a variety of factors including the activity of the specific compound employed, the age, body weight, general health, sex, diet, time of administration, route of administration, rate of excretion, drug combination, and the severity of the particular disorder or disease undergoing therapy. A therapeutically effective amount for a given situation can be readily determined by routine experimentation and is within the skill and judgment of the ordinary clinician.

The total daily usage of the compounds and compositions of the present disclosure may be decided by the attending physician within the scope of sound medical judgment. The specific therapeutically effective dose level for any particular patient may depend upon a variety of factors including the disease or disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; and drugs used in combination or coincidental with the specific compound employed.

Further reference is made to the following experimental examples.

### EXAMPLES

The following examples are given for the purpose of illustrating various embodiments of the invention and are not meant to limit the present disclosure in any fashion. The present examples, along with the methods described herein are presently representative of preferred embodiments, are provided only as examples, and are not intended as limitations on the scope of the invention. Changes therein and other uses which are encompassed within the spirit of the disclosure as defined by the scope of the claims will occur to those skilled in the art.

### Example 1

Intranasal Treatment of 5XFAD Mice with a p40₂ mAb Reduces the Load of Amyloid Plaque from the Hippocampus

Amyloid plaque is an important feature of AD pathology, which is modeled in 5XFAD mice. See Dinkins et al., “The 5XFAD Mouse Model of Alzheimer's Disease Exhibits an Age-Dependent Increase in Anti-Ceramide IgG and Exogenous Administration of Ceramide Further Increases Anti-Ceramide Titers and Amyloid Plaque Burden,” (2015) J Alzheimers Dis. 46(1): 55-61. Thus, it was examined whether treatment with p40₂ mAb at a very low dose via intranasal route is capable of reducing the load of amyloid plaques from the hippocampus of 5XFAD mice.

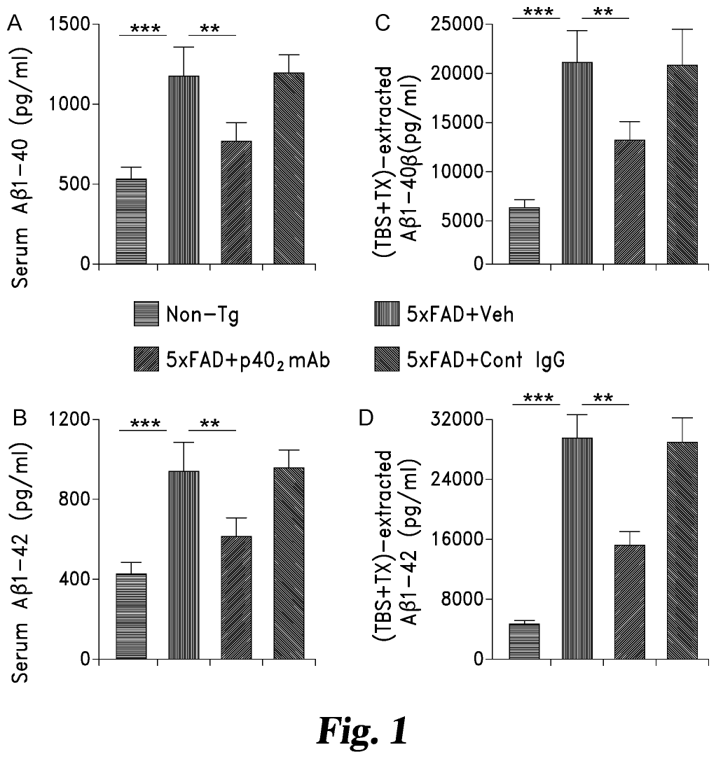

Six-month old 5XFAD Tg mice (n=6 per group) were treated with p40₂ mAb a3-1d at a dose of 25 ng/mouse intranasally once a week for one month. ELISA of serum (FIG. 1A, FIG. 1B) and TBS plus Triton X-100—extracted hippocampal fractions (FIG. 1C, FIG. 1D) revealed a marked increase in Aβ1-40 and Aβ1-42 in 5XFAD mice compared with non-Tg mice. However, a significant decrease in both Aβ1-40 and Aβ1-42 was seen with p40₂ mAb, but not control IgG, treatment (FIG. 1A, FIG. 1D).

These results suggest that intranasal immunotherapy with p40₂ mAb is capable of reducing Aβ burden in the hippocampus of 5XFAD mice and could be a useful treatment to reduce amyloid plaque burden in the brain as well as a possible treatment for Alzheimer's disease.

### Example 2

Intranasal Treatment of 5XFAD Mice with a p40₂ mAb Improves Memory and Spatial Learning

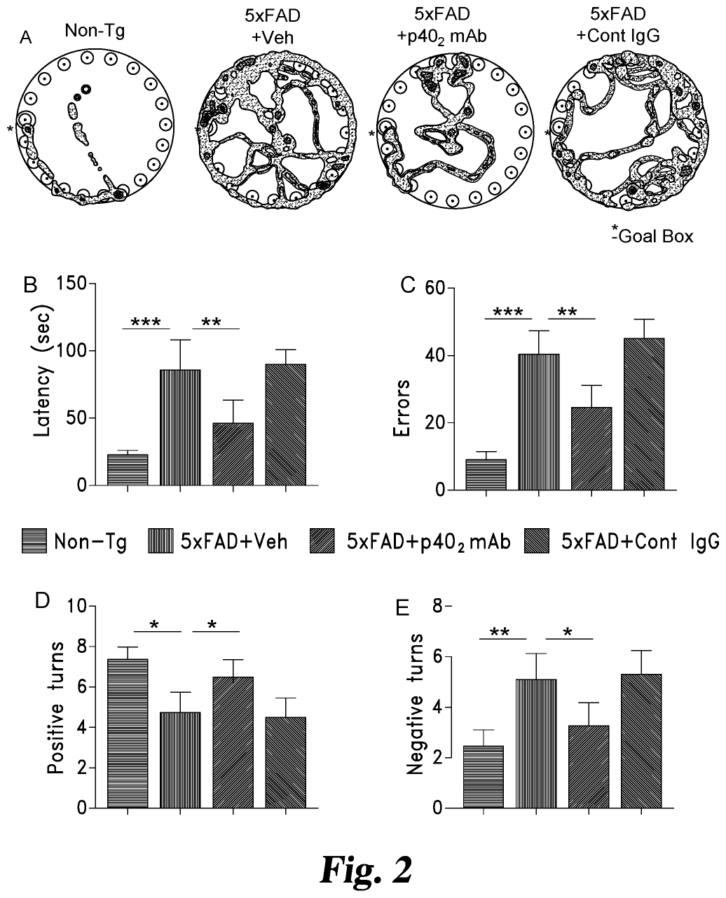

The eventual goal of neuroprotection in AD is to improve and/or protect memory. Major functions of the hippocampus are to generate and organize long-term memory and spatial learning. Therefore, it was examined whether weekly p40₂ mAb treatment also protected memory and learning in 5XFAD mice. Mice were monitored for spatial learning and memory by Barnes maze (FIG. 2A-E). Here, mice were trained for two consecutive days followed by examination on day 3. During training, the overnight food-deprived mouse was placed in the middle of the maze in a 10 cm high cylindrical start chamber. After 10 s, the start chamber was removed to allow the mouse to move around the maze to find out the color food chips in the baited tunnel. Significant cognitive impairment was seen in 5XFAD mice as compared to age-matched non-Tg mice (FIG. 2A-C). 5XFAD treated mice took a longer time to find the correct hole (FIG. 2A-B) and made more errors (FIG. 2C) as compared to non-Tg mice. However, weekly intranasal immunotherapy with p40₂ mAb significantly reduced latency and errors of 5XFAD mice in reaching the target hole (FIG. 2A-C). Similarly, the T-maze test also exhibited significant improvement in the performance of 5XFAD mice as shown by increase in the number of positive turns (FIG. 2D) and the reduction of negative turns (FIG. 2E).

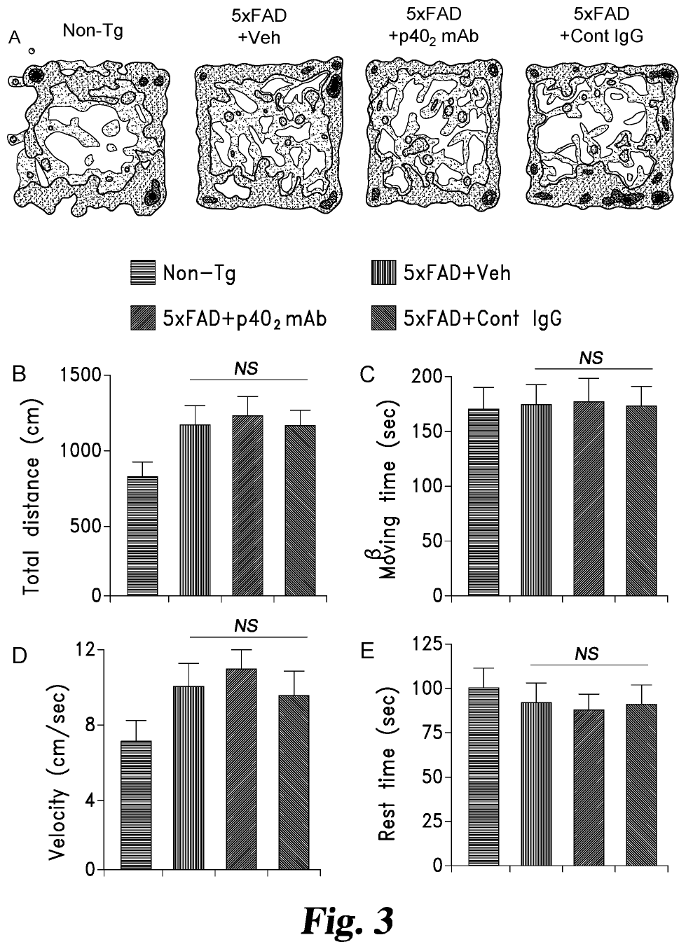

Since the decreased latency in either Barnes maze or T-maze test could be confounded with the increased locomotion of animals, the locomotor activity of these animals by open field was also examined and the results shown in FIG. 3A-D. No significant differences were observed in total distance moved (FIG. 3A, FIG. 3B), velocity (FIG. 3C) and rest time (FIG. 3D) in treated or untreated 5XFAD mice, nullifying the possibility of interference by increased locomotion in the hippocampus-dependent behaviors.

Thus, taken all together, the present results provide evidence that intranasal immunotherapy with neutralizing mAbs against IL-12 p40 homodimer may be beneficial for AD patients.

As will be appreciated from the descriptions herein, a wide variety of aspects and embodiments are contemplated by the present disclosure, examples of which include, without limitation, the aspects and embodiments listed below:

Methods and pharmaceutical compositions and/or formulations comprising administering to a patient in need thereof an effective amount of a pharmaceutical composition comprising of a pharmaceutical composition comprising p40₂ (mAb-p40₂ a3-1d) antibodies.

Methods and pharmaceutical compositions and/or formulations useful for the treatment of Alzheimer's disease and related disorders in which the amyloid deposition is present in the brain, comprising intranasally administering to a patient in need thereof an effective amount of a pharmaceutical composition comprising p40₂ (mAb-p40₂ a3-1d) antibodies.

Methods for the treatment for AD which includes administration to a patient of at least one monoclonal antibody against p40₂ (mAb-p40₂ a3-1d).

Methods for reducing the load of amyloid plaques in the brain is disclosed which includes administration to a patient of at least one monoclonal antibody against p40₂ (mAb-p40₂ a3-1 d).

Methods for a treatment for Alzheimer's disease is disclosed which includes administration to a patient of a combination of recombinant p40 and at least one monoclonal antibody against p40₂ (mAb-p40₂ a3-1d).

Methods for reducing the load of amyloid plaques in the brain is disclosed which includes administration to a patient of a combination of recombinant p40 and at least one monoclonal antibody against p40₂ (mAb-p40₂ a3-1d).

Methods and compositions wherein the pharmaceutical composition is administered to the patient by injection, orally, with a transdermal patch, and/or intranasally. In some embodiments, the pharmaceutical composition is preferably inhaled by the patient.

Methods wherein the pharmaceutical composition is formulated together with a pharmaceutically acceptable carrier or excipient.

Under the provisions of the Budapest Treaty on the International Recognition of the Deposit of Microorganisms for the Purpose of Patent Procedure, Applicant has deposited biological material comprising twenty-five (25) vials of hybridoma with the designation of antibody (mAb)a3-1d with the International Depositary Authority, American Type Culture Collection (ATCC) of 1080 University Blvd., Manassas, Va. 20110-2209, on Dec. 3, 2020 and assigned deposit number PTA-126900. The deposited hybidoma comprises a monoclonal antibody against the p40₂ homodimer which comprises mAb-p40₂ a3-1d.

While embodiments of the present disclosure have been described herein, it is to be understood by those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for treating an Alzheimer's disease in a patient, the method comprising: administering a combination of a recombinant p40 monomer protein and at least one monoclonal antibody against p40₂ homodimer to the patient, wherein the at least one monoclonal antibody against the p40₂ homodimer comprises mAb-p40₂ a3-1d.

2. The method of claim 1, wherein the recombinant p40 monomer protein is administered in a plurality of doses.

3. The method of claim 1, wherein the at least one monoclonal antibody against p40₂ homodimer is administered in a single dose.

4. The method of claim 1, wherein the recombinant p40 monomer is administered in a plurality of doses and the at least one monoclonal antibody against p40₂ homodimer is administered in a single dose.

5. The method of claim 1, wherein the administration ameliorates one or more of symptoms of Alzheimer's disease, the one or more symptoms comprising memory loss and/or learning deficits.

6. The method of claim 1, wherein the at least one monoclonal antibody against p40₂ homodimer is administered in a plurality of doses.

7. The method of claim 1, wherein the recombinant p40 monomer is administered in a plurality of doses and the at least one monoclonal antibody against p40₂ homodimer is administered in a plurality of doses.

8. The method claim 1, wherein the recombinant p40 monomer protein and/or at least one monoclonal antibody against p40₂ homodimer is administered to the patient intranasally.

9. A method for reducing the load of amyloid plaques treating an Alzheimer's disease in a patient, the method comprising: administering a combination of a recombinant p40 monomer protein and at least one monoclonal antibody against p40₂ homodimer to the patient, wherein the at least one monoclonal antibody against the p40₂ homodimer comprises mAb-p40₂ a3-1d.

10. The method of claim 9, wherein the recombinant p40 monomer protein is administered in a plurality of doses.

11. The method of claim 9, wherein the at least one monoclonal antibody against p40₂ homodimer is administered in a single dose.

12. The method of claim 9, wherein the recombinant p40 monomer is administered in a plurality of doses and the at least one monoclonal antibody against p40₂ homodimer is administered in a single dose.

13. The method of claim 9, wherein the administration ameliorates one or more of symptoms of increased amyloid plaques in brain tissue, the one or more symptoms comprising memory loss and/or learning deficits.

14. The method of claim 9, wherein the at least one monoclonal antibody against p40₂ homodimer is administered in a plurality of doses.

15. The method of claim 9, wherein the recombinant p40 monomer is administered in a plurality of doses and the at least one monoclonal antibody against p40₂ homodimer is administered in a plurality of doses.

16. The method claim 9, wherein the recombinant p40 monomer protein and/or at least one monoclonal antibody against p40₂ homodimer is administered to the patient intranasally.
